(19) United States
(12) Patent Application Publication
(10) Pub. No.: US 2008/0135170 A1
(43) Pub. Date: Jun. 12, 2008

(54) Systems and Methods for Laser Bonding Catheter Components

(76) Inventors: Fugui He, Pembroke Pines, FL (US); Scott B. Sidwell, Hollywood, FL (US); Thomas N. Trotta, Miami Shores, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/597,114

(22) PCT Filed: May 21, 2005
(86) PCT No.: PCT/US05/19247

§ 371 Date: Aug. 20, 2007

Related U.S. Application Data
(60) Provisional application No. 60/573,588, filed on May 21, 2004.

(51) Int. Cl. B29C 65/16; A61M 25/10

(52) U.S. Cl. 156/272.8; 604/103

(57) ABSTRACT

Systems and methods are described for laser bonding polymer catheter components by using laser energy to create localized heat fusion bonds between two or more polymer catheter components.

### CROSS-REFERENCE TO RELATED APPLICATION

This application claims the benefit of priority of United States Provisional Patent Application No. 60/573,588 filed on May 21, 2004.

### BACKGROUND AND SUMMARY OF THE INVENTION

1. Technical Background

The present invention relates generally to medical devices, and more particularly to laser bonding of medical catheter components.

2. Discussion

Many different medical therapeutic treatments are conducted using catheters and catheter systems of various kinds. Examples of different type of catheter include balloon catheters, diagnostic catheters, guiding catheters, stent delivery system catheters, injection catheters, gene therapy catheters, electrophysiology catheters, therapeutic drug delivery catheters, ultrasound catheters, laser angioplasty catheters, etc.

The components of medical catheters are often made of materials including polymers or plastics. Such polymer materials may be selected to have acceptable properties, including biocompatibility, pull strength, longitudinal or column strength, and bending flexibility. Some of the polymer materials may include polyamides, polyurethanes, nylons, polyethylenes, including high-density polyethylene (HDPE), polyether block amide (PEBA) which is available as Pebax®, polyester (PET) or polyetheretherketone (PEEK). Also, any of the catheter components may be made of a coextrusion or a blend or a block copolymer of such polymer materials.

Some medical catheters include polymer components that are affixed together. When polymer components are affixed together, the desirable properties of such a joint, seal or bond between components may include its strength, and its ability to hold high fluid pressures or to exhibit high pull strength. It is desirable to provide systems and methods for laser bonding catheter components effectively and efficiently.

An additional desirable feature of catheter component bonding techniques may include minimizing heating during formation of the bond, so as to minimize any adverse impact on flexibility which may occur during bonding. Another desirable feature may include creating a bond having as small a size as possible.

Previous methods for bonding polymer catheter components have included adhesives, thermal bonding techniques including the methods referred to as “hot jaws,” and laser bonding. An example of known technology involving laser bonding of catheter components is shown in the following patents: U.S. Pat. Nos. 5,267,959 and 5,501,759, both entitled “Laser Bonding of Angioplasty Balloon Catheters,” which issued to Forman on Dec. 7, 1993 and Mar. 26, 1996 respectively. These methods of laser bonding catheter components require that the wavelength of the laser be selected, and the polymer materials of the catheter components also be selected, such that all of the polymers have high absorption at the wavelength selected for the laser. In other words, the polymer materials of all catheter components to be laser bonded must be made of material having laser energy absorption curves that reach a sufficiently high absorption level at a common specific wavelength of the laser energy. According to this known method, the catheter components meeting these material requirements are placed in contact with each other, and a laser emitting energy at the common or overlapping wavelength is directed at the location where a bond is desired.

This requirement of an overlapping common wavelength for high absorption in all of the components to be bonded necessarily limits the types and varieties of materials that can be selected for a particular catheter. In addition, the entirety of each component is heated at the location where the laser energy is directed. Also, the entire thickness of the components is heated at the region where the laser energy is directed.

In the particular example of bonding together tubular component(s), with at least a portion of one component surrounding the other component, it is desirable to provide compression around the outer component during laser bonding. This compression encourages good contact between the components so the laser energy will form a strong bond. Accordingly, the compressive force is preferably uniform, and located at the site of the bond. Such compression may be applied by object(s) that are substantially transparent to the laser energy, which allow the laser beam to pass through without substantial resistance or heating.

Some prior systems for laser bonding catheter components involved using a piece of shrink tubing to apply the desired compressive force during laser bonding. However, a new piece of shrink tubing must be used for each unit, which may add undesirable cost to constructing the medical device. Also, it may be difficult to remove the shrink tubing from the catheter components after bonding.

It is thus desirable to form a laser bond having the desired features, between any two or more catheter components made of material(s) including at least one polymer, and where the method provides efficient and effective bonding.

It is also desirable to provide a repeatable source of compression at the desired site of the bond, which is transparent to the laser energy, as well as being easily removable. Another desirable feature is a compression source that is reusable, in other words that does not require a one-time use production item to be discarded each time a laser bond is made.

It would also be desirable to provide systems and methods for bonding polymer catheter components, in which the bonds are at least as strong as those available with known laser bonding techniques, yet which are even smaller in size. Another desirable feature would be the freedom to choose polymer materials having desirable performance characteristics, without being limited to polymers with high laser energy absorption at the selected wavelength. Indeed, it would be desirable to select the polymer materials and the laser wavelength without regard to the absorption of the materials at that wavelength.

The terms “high absorption” and “low absorption” refer to relative absorption of laser energy. They may be understood in terms of the percentage transmission of laser energy through a particular material, such that “high absorption” may refer to less than 50% transmission, and “low absorption” may refer to more than 50% transmission. As a matter of preference when selecting a material, a material may be selected for “high absorption” if it exhibits less than 25% transmission, and a material may be selected for “low absorption” if it exhibits greater than 75% transmission of laser energy.

By way of example, the present invention will be described in relation to a balloon catheter and methods of bonding a balloon component to one or more catheter shaft components. However, it should be understood that the present invention relates to any catheter or methods for bonding catheter components having the features recited in any one of the following claims, and is not limited to any particular treatment or type of catheter, or catheter construction, or the particular example embodiments described below.

Additional examples of different types of catheters may include balloon catheters, diagnostic catheters, guiding catheters, stent delivery system catheters, injection catheters, gene therapy catheters, electrophysiology catheters, therapeutic drug delivery catheters, ultrasound catheters, laser angioplasty catheters, etc. The laser bonding systems and methods of the present invention may be used with or applied to any suitable type of catheter or catheter components.

These and various other objects, advantages and features of the invention will become apparent from the following description and claims, when considered in conjunction with the appended drawings. The invention will be explained in greater detail below with reference to the attached drawings of a number of examples of embodiments of the present invention.

BRIEF DESCRIPTION OF THE DRAWINGS

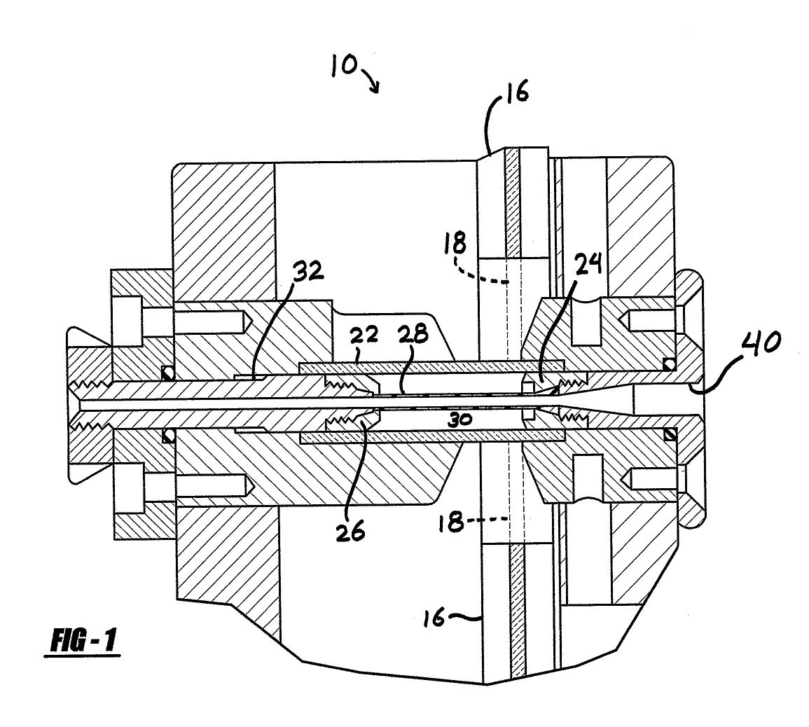

FIG. 1 is a partial cross-section view of an example system for laser bonding catheter components;

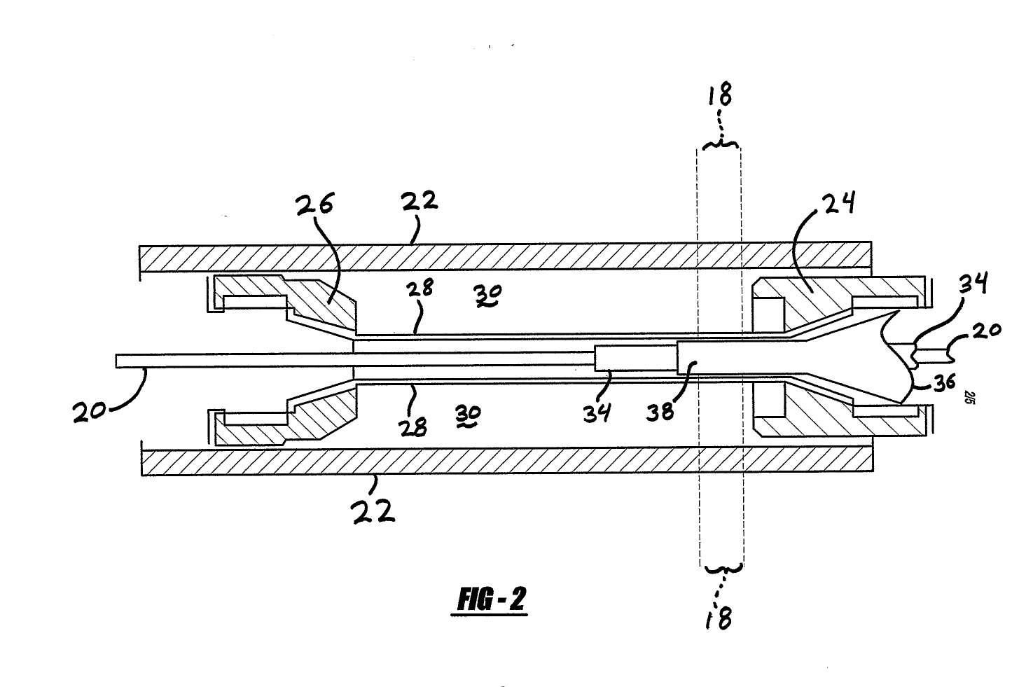

FIG. 2 is an expanded cross-section view of the example system for laser bonding catheter components of FIG. 1;

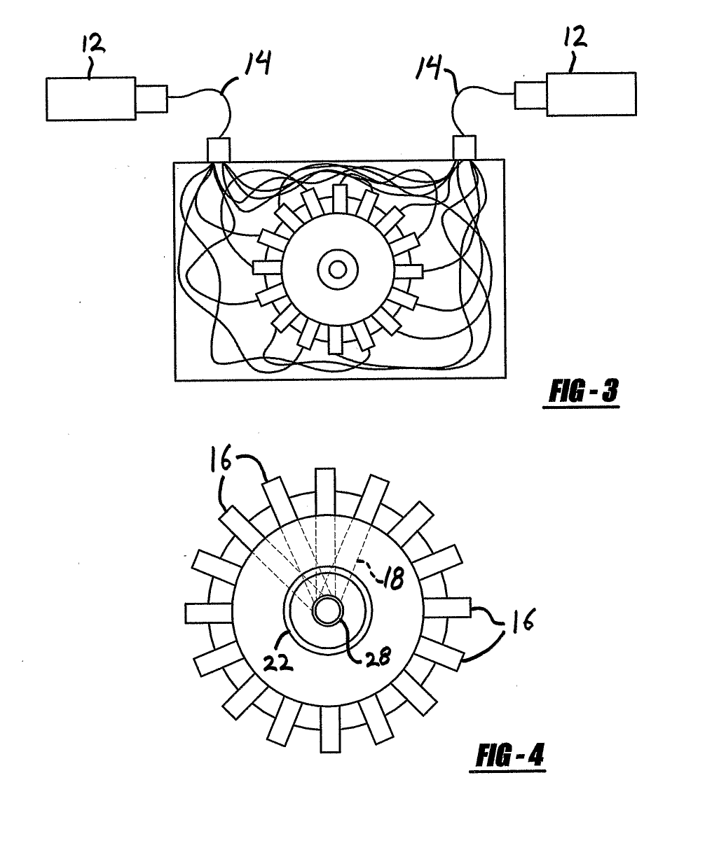

FIG. 3 is a diagrammatic view of another example system for laser bonding catheter components;

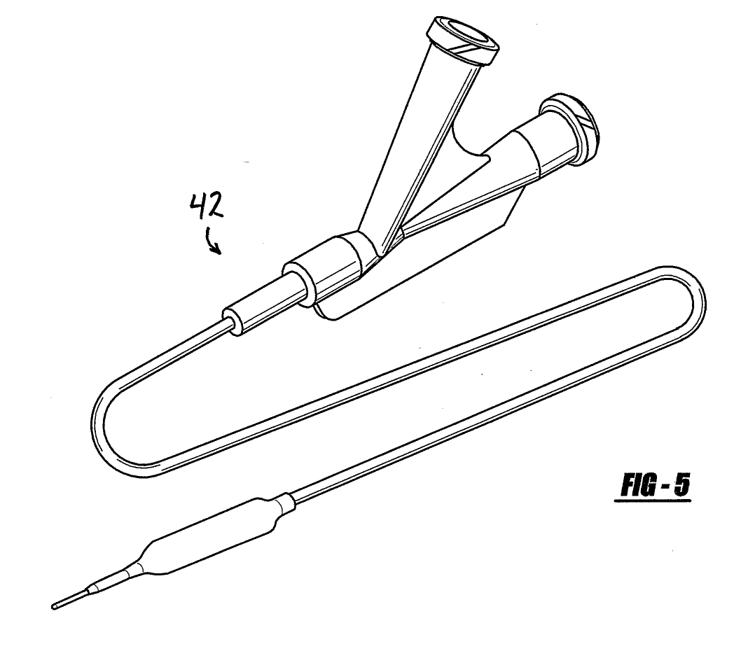

FIG. 4 is a diagrammatic view of the example system for laser bonding catheter components of FIG. 3; and

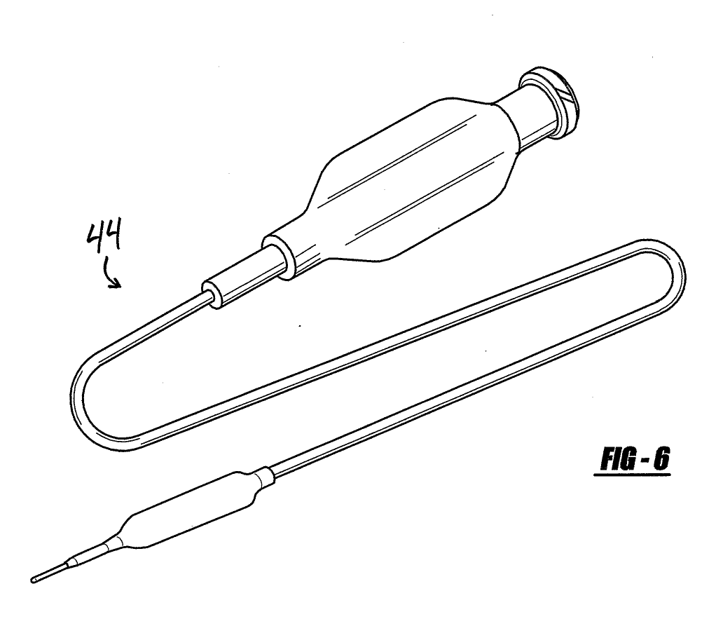

FIGS. 5 and 6 are perspective views of medical catheters having components bonded together by a laser system.

### DETAILED DESCRIPTION OF THE PREFERRED EMBODIMENTS

The following description of the preferred embodiments of the present invention is merely illustrative in nature, and as such it does not limit in any way the present invention, its application, or uses. Numerous modifications may be made by those skilled in the art without departing from the true spirit and scope of the invention.

According to one example of the present invention, a system 10 for laser bonding two or more catheter components is depicted in the drawings. The system 10 includes a laser generator 12, an optical fiber 14 or other element for transmitting the laser energy, and one or more emitters 16 for emitting the laser energy directed toward the catheter components to be bonded. A laser beam 18 is indicated with dashed lines. The system 10 may also include a mandrel 20 for supporting the catheter components.

A pressure chamber is defined by a tubular outer wall 22, a first and second end assembly 24 and 26, and a flexible compression tube 28. Together, the outer wall 22, first and second end assembly 24 and 26, and flexible compression tube 28 define an annular space 30. Annular space 30 can be selectively pressurized by a source of pressurized fluid communicating with the annular space 30 through passage 32.

The outer wall 22 and compression tube 28 are both made of materials that will allow laser energy to be transmitted to catheter components with very little resistance. In other words, they have very low absorption of laser energy at the selected wavelength(s), and consequently a very high percentage transmission of the laser energy. They may be made of any suitable material having such low absorption. For example, the outer wall 22 may be made of glass, and the compression tube 28 may be made of any suitable polymer, such as for example silicone. may also be any suitable fluid, which also has very low absorption of the laser energy, which may for example be air.

As an example, some catheter components are shown in the drawings, in this case a balloon catheter having a catheter shaft that includes a tubular inner body 34, and a balloon 36 having a balloon distal leg 38. In this particular example, the laser bonding system 10 is being used to bond the balloon distal leg 38 to the inner body 34.

The first end assembly of the pressure chamber defines an aperture 40 for receiving the catheter components that are to be laser bonded. Of course, the systems and the methods of the present invention may be used to laser bond any two or more catheter components, and is not limited to the particular types of catheter components shown in the drawings.

One example method of laser bonding using the system shown in FIG. 1 is as follows: providing a laser system 10 including a pressure chamber with a flexible compression sleeve 28, mandrel 20 is inserted within a lumen or passage defined by the inner body 34, and they are inserted into the balloon 36, such that balloon distal leg 38 is aligned with the portion of the inner body 34 where the bond is to be formed. The resulting assembly is inserted into the aperture 40 defined by the first end assembly, so that the laser emitter(s) are directed toward the selected bonding site. The pressure source pressurizes the annular space of the pressure chamber, thereby causing the flexible compression tube to flex radially inward and impose a compressive force on the balloon distal leg 38 and inner body 34. The laser system 10 is energized, to cause a laser beam(s) 18 to exit the emitter(s) 16, and to pass through the outer wall 22 and the compression tube 28 with low absorption and without substantially generating heat, and then generating heat to form a thermal fusion bond of the balloon distal leg 38 and inner body 34 at the desired bonding site.

Thereafter, the laser system 10 is deactivated and the pressure is relieved, thereby relieving the compressive force of the compression sleeve 28. The catheter components are then removed from the aperture 40, and the process may be repeated with a new set of catheter components.

Of course, various arrangements of laser systems may be used. For example, only one laser emitter may be provided, in which case the emitter may move around the catheter components or the catheter components may be moved or rotated under the laser beam.

Another example of a laser system is shown in FIGS. 3 and 4, having multiple emitters. The emitters may be arranged to generate an overlapping pattern of laser beams, as shown in FIG. 4. The angle between adjacent laser emitters may be 22.5 degrees, such that the laser beams overlap a catheter component subassembly such that any point in the heating area may simultaneously receive laser energy from three laser emitters.

Additional types of laser systems may include (i) a laser system having a single wavelength which exhibits high absorption in the material of an inner catheter component, or in the materials of all the catheter components; (ii) a laser system having a single wavelength which exhibits high absorption of a color of one or more of the catheter component(s), or of an ink or other colorant applied to one or more of the catheter component(s); or (iii) a laser system having at least one emitter which emit at least one wavelength, each selected to have high absorption of one of the catheter components, which wavelengths need not overlap. In this third type of system, the materials may thus be selected freely, without regard to whether they have high absorption in a single wavelength.

One particular type of laser which may be used with various polymers is the diode laser. Diode lasers are preferred for the laser bonding systems and methods of the present invention because their operating wavelengths are within the low energy absorption range (and high percentage transmission range) of most plastics and polymers.

The flexible compression tube offers a repeatable source of uniform compression, which releasably enables easy removal of the catheter components from the laser bonding system, without a one-time piece of shrink tube that must be discarded with each bonding process. Also, the compression force and laser heating energy are independent, allowing them to be individually optimized.

FIGS. 5 and 6 show catheters having components bonded together by a laser system according to the present invention, including a balloon catheter 42 having an “over-the-wire” arrangement and a balloon catheter 44 having a “rapid exchange” configuration.

It should be understood that an unlimited number of configuration for the present invention cold be realized. The foregoing discussion describes merely exemplary embodiments illustrating the principles of the present invention, the scope of which is recited in the following claims. Those skilled in the art will readily recognize from the description, claims and drawings that numerous changes and modifications can be made without departing from the spirit and scope of the invention.

What is claimed is:

1. A method for laser bonding catheter components, comprising the steps of:
(a) providing a laser system including at least one emitter capable of selectively generating a laser beam having a specific wavelength;
(b) providing a pressure chamber, defined by a tubular outer wall, a first and second end assembly, and a flexible compression tube positioned inside the outer wall; the pressure chamber defining an annular space; and at least the first end assembly defining an aperture;
and providing a source of pressurized fluid for selectively pressurizing the annular space defined by the pressure chamber;
wherein the at least one emitter of the laser system is directed at a selected region area of the compression tube;
wherein the outer wall and compression tube are made of one or more materials having relatively low absorption of laser energy at the specific wavelength;
(c) providing a first and second tubular catheter component, each component being made of materials including at least one polymer; and placing the first and second catheter components into physical contact with each other to define a contact area;
and inserting the first and second catheter components into the aperture defined by the first end assembly, so that the at least one laser emitter is directed toward a selected bonding site in the contact area;
wherein at least one of the first and second components has relatively high absorption of laser energy at the specific wavelength;
(d) activating the source of pressurized fluid to pressurize the annular space defined by the pressure chamber, thereby causing the flexible compression tube to flex radially inward and impose a compressive force on the first and second catheter components;
(e) energizing the laser system to cause a laser beam to exit the at least one emitter, and to pass through the outer wall and the compression tube with low absorption and without substantially generating heat, and then generating heat to form a thermal fusion bond of the first and second catheter components;
(f) deactivating the laser system; and relieving the pressure in the pressure chamber, thereby relieving the compressive force; and
(g) removing the bonded catheter components from the aperture of the first end assembly of the pressure chamber.

2. The method of claim 1, further comprising the additional step following step (g) of: repeating the steps (a)-(g) with a second pair of first and second catheter components, wherein the laser system and pressure chamber are ready for repeating such steps immediately after step (g), and wherein the same compression tube is used with successive pairs of first and second catheter components.

3. The method of claim 1, further comprising the additional step following step (e) of: moving or rotating one or both of first end assembly and the at least one emitter, to cause relative rotation of the first and second catheter components with respect to the at least one emitter.

4. The method of claim 1, further comprising multiple emitters, each arranged to define a corresponding bonding area, the emitters being collectively arranged so that their respective bonding areas are adjacent or overlapping.

5. The method of claim 1, wherein the pressurized fluid is air.

6. The method of claim 1, wherein at least a portion of the second catheter component surrounds at least a portion of the first catheter component.

7. The method of claim 6, wherein the first catheter component polymer material has high absorption at the specific wavelength, and the second catheter component polymer material has low absorption at the specific wavelength, such that the bond is formed in the contact area between the first and second catheter components.

8. The method of claim 1, wherein the polymer materials of the first and second catheter components both have high absorption at the specific wavelength.

9. The method of claim 1, wherein the laser system further comprises a second emitter capable of selectively generating a laser beam having a second specific wavelength;
wherein the polymer material of the first catheter component has high absorption at the first specific wavelength, and the polymer material of the second catheter component has high absorption at the second specific wavelength.

10. The method of claim 1, wherein the material of the compression tube is silicone.

11. The method of claim 1, wherein the laser system is a diode laser.

12. A medical catheter having laser bonded catheter components, made by a process comprising the steps of:
(a) providing a laser system including at least one emitter capable of selectively generating a laser beam having a specific wavelength;
(b) providing a pressure chamber, defined by a -tubular outer wall, a first and second end assembly, and a flexible compression tube positioned inside the outer wall; the pressure chamber defining an annular space; and at least the first end assembly defining an aperture;
and providing a source of pressurized fluid for selectively pressurizing the annular space defined by the pressure chamber;
wherein the at least one emitter of the laser system is directed at a selected region area of the compression tube;
wherein the outer wall and compression tube are made of one or more materials having relatively low absorption of laser energy at the specific wavelength;
(c) providing a first and second tubular catheter component, each component being made of materials including at least one polymer; and placing the first and second catheter components into physical contact with each other to define a contact area;
and inserting the first and second catheter components into the aperture defined by the first end assembly, so that the at least one laser emitter is directed toward a selected bonding site in the contact area;
wherein at least one of the first and second components has relatively high absorption of laser energy at the specific wavelength;
(d) activating the source of pressurized fluid to pressurize the annular space defined by the pressure chamber, thereby causing the flexible compression tube to flex radially inward and impose a compressive force on the first and second catheter components;
(e) energizing the laser system to cause a laser beam to exit the at least one emitter, and to pass through the outer wall and the compression tube with low absorption and without substantially generating heat, and then generating heat to form a thermal fusion bond of the first and second catheter components;
(f) deactivating the laser system; and relieving the pressure in the pressure chamber, thereby relieving the compressive force; and
removing the bonded catheter components from the aperture of the first end assembly of the pressure chamber.
